(19) United States
(12) Patent Application Publication
(10) Pub. No.: US 2010/0048505 A1
(43) Pub. Date: Feb. 25, 2010

(54) Composition Having Effect of Treating, Preventing, or Improving Diabetes or Diabetic Complication and Drink Comprising the Same

(76) Inventors: Shigeyoshi Fujii, Tokyo (JP); Izumi Takao, Tokyo (JP); Li-kun Han, Toyama (JP); Asako Ishii, Kumamoto (JP); Hiromichi Okuda, Ehime (JP)

Correspondence Address: FITCH EVEN TABIN & FLANNERY, 120 SOUTH LASALLE STREET, SUITE 1600, CHICAGO, IL, 60603-3406, US

(21) Appl. No.: 12/374,021

(22) PCT Filed: Jul. 20, 2007
(86) PCT No.: PCT/US07/73977

§ 371 Date: Oct. 12, 2009

(30) Foreign Application Priority Data
Jul. 21, 2006 (JP) 2006-199060

(51) Int. Cl. A61K 31/715; C07H 99/00; A61P 3/10

(52) U.S. Cl. 514/54; 536/123.1

(57) ABSTRACT

A safe, economical, and convenient food drink having the effect of treating, preventing, or improving diabetes or diabetic complications without involving significantly changed dietary life habits is provided. A food or drink is provided for treating, preventing, or improving diabetes or diabetic complications, comprising oligosaccharides at a concentration of 0.15 to 10% by weight wherein the oligosaccarides include mannose molecules linked together with degree of polymerization of 2 to 10 (inclusive).

### FIELD OF THE INVENTION

The present invention relates to a composition having the effect of treating, preventing, or improving diabetes or diabetic complications, comprising oligosaccharides comprising mannose as a constituent sugar, and to a food or drink comprising the composition. The present invention also relates to effective use of unused resources.

### DESCRIPTION OF THE RELATED ART

Almost all of coffee extracted residues have previously been burned up, or treated as an industrial waste. Some coffee extracted residues have more recently become used as a compost raw material or an active carbon raw material, which is, however, less than sufficient in view of advanced utilization of unused resources; therefore, establishing further methods for advanced utilization of coffee extracted residues represents an important challenge.

Lifestyle-related diseases such as diabetes, hypertension, and hyperlipemia, which are rapidly increasing in recent years, are considered to be closely related to dietary life, and emphasis is placed on the prevention thereof by improved dietary life. The current status of the incidence of diabetes is particularly serious; one out of every six adults in Japan is estimated to correspond to sufferers and potential sufferers thereof. Type 2 diabetes, which accounts for 90% or more of diabetes, occurs in association with a reduction of insulin action being induced by reduced insulin secretion from pancreatic β-cells and lowered insulin sensitivity in target organs therefor together to produce hyperglycemia. In addition, once hyperglycemia occurs, there is a further insulin resistance due to glucose toxicity, resulting in the creation of a vicious circle. Diabetes seldom produces subjective symptoms at the early stages; thus, this disease often also leads to serious complications such as retinopathy, nephropathy, and neuropathy supervened because of the development thereof. Insulin resistance-improving agents such as thiazolidine derivatives are used as therapeutic agents therefor, but have been also reported to produce side effects due to long-term use. Accordingly, it is probably important to, through the daily diet, not only prevent the onset of diabetes but also suppress and improve the development thereof in a still mild state even after the onset, which corresponds to preventive medicine. This shows that it is very important to suppress and prevent insulin resistance representing a critical cause of diabetes and hyperglycemia at the early stages of the onset of diabetes. Thus, searches for substances having the effect of treating and preventing diabetes and studies on action mechanisms thereof have taken place; the diabetes-treating and -preventing effect of soybean isoflavone as a food-derived ingredient has been reported. However, the efficacy of the isoflavone is low, and concerns exist about an affect of excess ingestion thereof on hormone balance. The present applicants have previously found that an intestine-regulating function accompanied by an excellent bifidobacterium-growing activity, a function improving serum lipid levels, and the like are exhibited by mannooligosaccharides with a degree of polymerization of 1 to 10 (inclusive) whose sugar chains have a low content of sugar residues other than a mannose residue and which are obtained from a mannan-enriched food material, mainly hydrolysate of extracted coffee residue. See Japanese Patent Laid-Open No. 2003-000211, Japanese Patent Laid-Open No. 2003-000196, and Japanese Patent Laid-Open No. 2003-286166, all of which have been incorporated by reference.

Prior to this time of this invention, however, it was been totally unknown whether the effect of treating, preventing, or improving diabetes or diabetic complications is present in the mannooligosaccharides or not.

The present invention provides a safe, economical and simple food and/or drink having the effect of treating, preventing, or improving diabetes or diabetic complications without involving significantly changed dietary life habits.

### SUMMARY OF THE INVENTION

As a result of intensive studies for solving the above-described problems, the present inventors have found that the effect of treating, preventing, or improving diabetes or diabetic complications is possessed by mannooligosaccharides whose sugar chains have a low content of sugar residues other than a mannose residue and which have a degree of polymerization of 2 to 10 (inclusive), or mannooligosaccharides in each of which 2 to 10 (inclusive) mannose molecules are linked together, obtained from a mannan-enriched food material, mainly hydrolysate of extracted coffee residue, thereby accomplishing the present invention. In addition, it has been found that color- and acid-free mannooligosaccharides whose sugar chains have a low content of sugar residues other than a mannose residue and which have a degree of polymerization of 2 to 10 (inclusive) can be obtained to dramatically widen the scope of application of the mannooligosaccharides to food products.

Thus, various embodiments of the present invention include, but are not limited to, the following:

1. A food or drink for treating, preventing, or improving diabetes or diabetic complications, comprising oligosaccharides in each of which 2 to 10 (inclusive) mannose molecules are linked together at a concentration of 0.15 to 10% by weight;

2. A food or drink for suppressing an elevation of blood glucose level during glucose loading, comprising oligosaccharides in each of which 2 to 10 (inclusive) mannose molecules are linked together at a concentration of 0.15 to 10% by weight;

3. A food or drink for lowering blood glucose level, comprising oligosaccharides in each of which 2 to 10 (inclusive) mannose molecules are linked together at a concentration of 0.15 to 10% by weight;

4. A food or drink for improving insulin resistance, comprising oligosaccharides in each of which 2 to 10 (inclusive) mannose molecules are linked together at a concentration of 0.15 to 10% by weight;

5. The food or drink as described in any one of embodiments 1 to 4 above, wherein the oligosaccharides are oligosaccharides in each of which the number of mannose units in the molecule is 2 to 6;

6. The food or drink as described in any one of embodiments 1 to 5 above, wherein the oligosaccharides are ones obtained by subjecting mannan to hydrolysis treatment;

7. The food or drink as described in embodiment 6 above, wherein the mannan is obtained from coffee beans and/or coffee extracted residues;

8. The food or drink as described in any one of embodiments 1 to 7 above, wherein the oligosaccharides are β-1,4-mannooligosaccharides;

9. The drink as described in any one of embodiments 1 to 8 above, wherein the drink is one of a liquid coffee drink, a liquid tea drink, a liquid fruit juice drink, an instant coffee, a powdered coffee mix drink, a powdered tea drink, and a powdered fruit juice drink; and

10. Methods for treating, preventing, or improving diabetes or diabetic complications in a subject, comprising administering an effective amount of the oligosaccharides as described in any of embodiments 1 to 9 above.

For purposes of this invention, the concentration of 0.15 to 10% by weight is intended to be the concentration in the food or drink product in its final form and ready for consumption by the consumer. Thus, for a dry powdered instant beverage composition intended to be reconstituted in water, the concentration would refer to the concentration in the reconstituted aqueous beverage.

Addition of mannooligosaccharides having the effect of treating, preventing, or improving diabetes or diabetic complications to a food and drink enables the oligosaccharides to be readily and economically ingested in daily dietary life, permitting the effect of treating, preventing, or improving diabetes or diabetic complications to be expected. The mannooligosaccharides having the effect of treating, preventing, or improving diabetes or diabetic complications are also available from wastes such as coffee extracted residues; thus, previously unused resources can be also effectively utilized. Preferably, the final compositions containing the mannooligosaccharides are in the form of drinks or beverages.

### BRIEF DESCRIPTION OF THE DRAWINGS

FIG. 1 illustrates the results of the glucose tolerance test discussed in Example 1 for mice ingesting a high-fat diet.

FIG. 2 illustrates the results of the glucose tolerance test discussed in Example 2 for diabetic rats.

### DETAILED DESCRIPTION

The content of the present invention will now be described in detail. For the purpose of the present invention, the term “mannooligosaccharides” refers to oligosaccharides comprising the monosaccharide mannose as a constituent. As used herein, the term “oligosaccharides” generally refers to substances which fall on between monosaccharides and polysaccharides and comprise the glycosyl bonds of a certain small number of monosaccharide molecules. In other words, the oligosaccharides are polymers each having a relatively small number of monosaccharide molecules linked together. The term “oligosaccharides” means a composition comprising a plurality of oligosaccharide molecules each composed of various numbers of constituent monosaccharides. The term “mannooligosaccharides” refers to a composition comprising a plurality of oligosaccharides each composed of various numbers of constituent monosaccharides.

The degree of polymerization or “DP” of an oligosaccharide means the number of monosaccharides constituting an oligosaccharide. Thus, the DP of an oligosaccharide as the monosaccharide mannose is expressed as “DP 1”, and the degree of polymerization of a mannooligosaccharide formed from 4 mannose molecules is 4 and therefore expressed as DP 4. Thus, it will be appreciated that the phrase “mannooligosaccharides in each of which 2 to 10 (inclusive) mannose molecules are linked together” means a composition of oligosaccharides having degrees of polymerization of 2 to 10 (inclusive).

The mannooligosaccharides used in the present invention are preferably a composition of plural types of oligosaccharides in each of which 2 to 10 (inclusive) mannose molecules are linked together. Particularly preferred oligosaccharides are a composition of oligosaccharides in each of which 2 to 6 inclusive mannose molecules are linked together.

One aspect of the present invention is a food, drink, or like product comprising a composition comprising the above-described mannooligosaccharides, at a concentration of 0.15 to 10% by weight, having the effect of treating, preventing, or improving diabetes or diabetic complications. Here, the phrase “composition having the effect of treating, preventing, or improving diabetes or diabetic complications” broadly and generally refers to a composition that, when ingested in sufficient amounts by a subject, has one or more effects including treating, preventing, or improving diabetes or diabetic complications in the subject, suppressing elevation of blood glucose levels in the subject during glucose loading, lowering blood glucose levels in the subject, and/or improving insulin resistance in the subject. Thus, a composition having the effect of treating, preventing, or improving diabetes or diabetic complications can be produced using such oligosaccharides.

Another aspect of the present invention is a food or drink comprising a composition having the effect of treating, preventing, or improving diabetes or diabetic complications, the drink having the effect of treating, preventing, or improving diabetes or diabetic complications in humans. The phrase “comprising mannooligosaccharides at a concentration of 0.15 to 10% by weight” means that the concentration of mannooligosaccharides in the food or drink is 0.15 to 10% by weight when the food or drink is, for example, a ready-to-eat food or a ready-to-drink beverage (e.g., liquid coffee drink, a liquid tea drink, or a liquid fruit juice drink. When a dry powdered composition (e.g., instant coffee, a powdered coffee mix, or a powdered fruit juice) is to be used to prepare a drink, this phrase also means that the concentration of mannooligosaccharides in the prepared drink obtained by the dissolution is 0.15 to 10% by weight.

The present invention also provides methods for treating, preventing, or improving diabetes or diabetic complications in a subject, especially humans, wherein such mannooligosaccharide-containing compositions are administered to the subject in a amount effected to treat, prevent, or improve diabetes or diabetic complications in the subject. Preferably the administration is oral and the mannooligosaccharide-containing compositions are in the form of a food or drink suitable for consumption by the subject. Drinks or beverages are the most preferred form for the mannooligosaccharide-containing compositions.

The mannooligosaccharides used in the present invention can be produced by hydrolyzing mannan, followed by extracting soluble solids. Here, the raw material mannan can be obtained by extraction, for example, from a coprameal of a coconut palm or flake, Huacra Palm of a South African Arecaceae (Palmae) plant, Chinese yam mannan, and yam mannan. The mannan thus obtained can be treated using at least one method selected from acid hydrolysis, high temperature thermal hydrolysis, enzymatic hydrolysis, and microbial fermentation; preferably the treated mannan is purified using a method such as active carbon treatment, adsorbent resin treatment, ion-exchange resin treatment, and ion-exchange membrane treatment to provide a sugar mixture. The sugar mixture comprises the above-described mannooligosaccharides having the effect of treating, preventing, or improving diabetes or diabetic complications. Thus, the composition thus obtained represents a composition having the effect of treating, preventing, or improving diabetes or diabetic complications according to the present invention. In addition, the composition having the effect of treating, preventing, or improving diabetes or diabetic complications according to the present invention may be one produced by treating glucomannan contained in Amorphophallus konjak, lily, narcissus, cluster amaryllis, or the like, or galactomannan contained in locust bean gum, guar gum, or the like, using at least one method selected from acid hydrolysis, high temperature thermal hydrolysis, enzymatic hydrolysis, and microbial fermentation, followed by separation and purification employing a method such as active carbon treatment, adsorbent resin treatment, ion-exchange resin treatment, and ion-exchange membrane treatment to increase the percentage of mannose as a constituent sugar.

Thus, mere “mannan” herein shall include, in its broad sense, galactomannan or glucomannan which is a polysaccharide having mannose and galactose or glucose as the constituent units, in addition to mannan which is a polysaccharide having only d-mannose as the constituent unit. D-Mannose is an aldohexose and differs from d-glucose only in having the opposite configuration of the hydroxyl group bonded to the carbon adjacent to the carboxyl group.

Further, the composition having the effect of treating, preventing, or improving diabetes or diabetic complications according to the present invention can be obtained by treating green coffee beans or roasted coffee beans using at least one method selected from acid hydrolysis, high temperature thermal hydrolysis, enzymatic hydrolysis, and microbial fermentation, followed by purification employing a method such as active carbon treatment, adsorbent resin treatment, ion-exchange resin treatment, and ion-exchange membrane treatment.

Alternatively, the composition can be also obtained by treating spent coffee residues using at least one method selected from acid hydrolysis, high temperature thermal hydrolysis, enzymatic hydrolysis, and microbial fermentation to produce an aqueous solution, followed by purification of the solution employing a method such as active carbon treatment, adsorbent resin treatment, ion-exchange resin treatment, or ion-exchange membrane treatment. In general, when roasted and ground coffee is extracted using a commercial extractor, at the time, galactose as a side chain of galactomannan contained in the roasted coffee is solubilized and arabinogalactan therein is solubilized by hydrolysis. Thus, mannan is abundantly present in coffee extracted residues and presumably assumes a straight-chain structure. On the other hand, cellulose is hardly decomposed and remains as a residue, but conditions of specifically hydrolyzing mannan without decomposing cellulose can be properly selected before extraction to provide the desired mannooligosaccharides.

Particularly, methods for decomposing coffee extracted residues include, but are not limited to, a method involving hydrolysis by acid and/or high temperature, a method involving decomposition by an enzyme, and a method involving decomposition by microbial fermentation. The method involving hydrolysis by acid and/or high temperature is disclosed, for example, in Japanese Patent Laid-Open Nos. 61-96947 and 02-200147, which are hereby incorporated by reference. Spent coffee residues coming out of a commercial multistage coffee extraction system may be hydrolyzed either by addition of an acid catalyst in a reaction vessel, or by short-time high-temperature treatment without addition of the acid catalyst. It is convenient to use a tubular plug flow reactor, but a good result can be obtained using any reactor provided that the reactor is suitable to conduct a short time reaction at a relatively high temperature. The reaction time and reaction temperature can be controlled for solubilization and hydrolysis to decompose mannan having DP 10 to 40 into mannooligosaccharides having DP 2 to 10, followed by separating the coffee residues to extract mannooligosaccharides. Here, the term “coffee extracted residues” refers to a so-called coffee extraction cake obtained after the extraction of roasted and ground coffee with a solvent such as water in the air or under conditions of applied pressure.

When the composition having the effect of treating, preventing, or improving diabetes or diabetic complications according to the present invention is obtained by subjecting, to hydrolysis treatment, coffee beans (including roasted coffee beans and roasted and ground coffee beans) and/or coffee extracted residues, the kind and growing area of the coffee beans used is not particularly limited. Coffee beans of any kind such as Arabica, Coffea Robusta, and Coffea Liberica coffee beans and coffee beans from any growing area such as Brazil and Colombia may be used; these kinds of beans may be used alone or in a blend of two or more thereof. Even low-quality or undersized coffee beans as generally condemned as having no commercial value may be used. There can be used coffee beans obtained by roasting the above-described coffee beans to a light, cinnamon, medium, or city roast using a roaster (such as direct-fired type, hot air type, far infrared-ray type, and charcoal-fired type roasters), and roasted and ground coffees (including ones in various forms such as coarse ground, medium-coarse ground, medium ground, and fine ground forms) obtained by grinding the above-described roasted coffee beans employing a common grinder, roll mill, or the like.

In addition, the coffee extracted residues used may be any coffee extracted residues, obtained after extraction either at ordinary pressure or under higher pressure, or from coffee of any origin or preparation method, provided that the residues are those obtained after subjecting the roasted and ground coffee to extraction treatment in the typical production process of a liquid coffee or instant coffee.

Some examples of the above-described hydrolysis treatments will now be described in detail. The method for decomposition by an enzyme may involve, for example, suspending coffee extracted residues in an aqueous medium, to which commercially available cellulase, hemicellulase, and the like are then, for example, added, followed by suspending the mixture while stirring. The conditions such as the amount of the enzyme and the acting temperature may be any such conditions used for conventional enzymatic reactions, and may be properly selected depending on conditions such as the optimal acting amount of, and the optimal temperature for, the enzyme used and other factors. The method for decomposition by microbial fermentation may involve, for example, inoculating a microbe producing cellulase, hemicellulase, and the like coffee extracted residues suspended in an aqueous medium for culture. The microbe used may be any microbe such as bacteria and basidiomycetes provided that it produces enzymes decomposing mannan in coffee extracted residues, and culture conditions and the like may be properly selected depending on the microbe used.

The reaction solution comprising mannooligosaccharides obtained by the above-mentioned methods, which contains a composition having the effect of treating, preventing, or improving diabetes or diabetic complications, may be subjected to purification as needed. Examples of the purification method include decolorization and deodorization using, for example, bone black, active carbon, a carbonation process, an adsorbent resin, a magnesia process, or a solvent extraction process, followed by desalting and deacidification employing, for example, an ion-exchange resin, an ion-exchange membrane, or electrodialysis. The combination of purification processes and the purification conditions may be properly selected depending on the amount of coloring matter, salts, acids, and the like in the reaction solution containing the mannooligosaccharides, and other factors.

The present invention also relates to an orally ingestible composition (e.g., a food or drink and more preferably a drink), comprising the above-described composition having the effect of treating, preventing, or improving diabetes or diabetic complications in humans or animals in accordance with the invention. In addition, the composition having the effect of treating, preventing, or improving diabetes or diabetic complications according to the present invention can be used in a wide range of fields including, but not limited to, drinks, foods, cosmetics, medicines, feeds, and the like. The composition having the effect of treating, preventing, or improving diabetes or diabetic complications adopted in the present applied invention may be administered in the form of a therapeutic and prophylactic preparation for diabetes or diabetic complications as a drug or a quasi drug. Preferably, the composition may be also administered in the form of a pharmaceutical composition producible by a well-known method. Examples of the pharmaceutical composition include tablets, capsules, powders, granules, solutions, and syrups. The composition having the effect of treating, preventing, or improving diabetes or diabetic complications according to the present invention is orally ingested particularly in the form of a food and/or drink by a human to exert the effect of treating, preventing, or improving diabetes or diabetic complications. The intake or dose of the composition for exerting the efficacy of the present applied invention is not particularly limited and may be properly changed depending on the body weight and age of takers or patients, the type and symptom of diseases, as well as the response of the individual to the composition. Generally, the composition may be used effectively in the range of 0.1 g to 40 g, preferably 0.5 g to 20 g per day for an adult.

Thus, when the mannooligosaccharides are ingested in the form of a drink, for example, assuming that the drink is ingested in an amount of 300 milliliters per day, the drink of the present invention should comprise the mannooligosaccharide composition at a drinking concentration of about 0.03 to 13% by weight, preferably 0.15 to 10% by weight. For offering the effect of treating, preventing, or improving diabetes or diabetic complications, the mannooligosaccharides are required to be ingested on the order of 0.5 to 20 g per day per person. With the assumption that 100 g of the drink of the present invention is ingested three times a day, a draft of the drink preferably comprises about 0.17 to 6.67 g of the mannooligosaccharides. For example, the amount of mannooligosaccharides contained per serving of a conventional instant coffee is about 0.02 to 0.1 g and extremely smaller than that of the drink according to the present invention, failing to produce the effect of treating, preventing, or improving diabetes or diabetic complications. Thus, the mannooligosaccharides obtained by the foregoing method can be added to a drink to produce a mannooligosaccharides-enriched drink.

In ingesting such a drink, it is very preferable that the mannooligosaccharide composition can be dissolved in water or the like to prepare the drink instantly on the spot. Thus, the present invention also relates to a powdered drink mix comprising 0.15 to 20 g of a composition comprising oligosaccharides in each of which 2 to 10 (inclusive) mannose molecules are linked together and 0.1 to 10 g of a drink raw material selected from the group consisting of a powdered coffee, a tea leaf, a powdered tea, and a powdered fruit juice. The powdered drink mix comprising mannooligosaccharides producing the effect of treating, preventing, or improving diabetes or diabetic complications is also preferable in terms of product preservation. Examples of such a powdered drink mix include instant coffees typified by instant coffee, tea leaves typified by black tea, green tea and oolong tea leaves, powdered teas obtained by drying tea drinks, and powdered fruit juices. When mixed with an instant coffee, the mannooligosaccharides are preferably used, for example, in an amount of 0.15 to 20 g based on 1.5 to 2.0 g of the instant coffee. Likewise, it is preferable that the mannooligosaccharides are mixed in an amount of 0.15 to 20 g based on 0.1 to 1.0 g of the powdered tea, or in an amount of 0.15 to 20 g based on 4.0 to 10 g of the powdered fruit juice. The instant coffee, powdered tea, and powdered fruit juice can be properly produced by conventional techniques. The powdered drink mix of the present invention may also contain, as needed, additives including a sweetener, a perfume, a food color, a thickener, a foaming agent, an emulsifier, a pH adjustor, and a fat and oil such as vegetable oil or milk fat.

According to the present invention, the composition having the effect of treating, preventing, or improving diabetes or diabetic complications prepared so as to contain oligosaccharides at high purity by hydrolyzing coffee extracted residues by an acid and/or heating may be directly added to a liquid coffee, an instant coffee, or the like for use; however, the addition of the composition subjected, as needed, to purification treatments such as decolorization, deodorization, and deacidification using active carbon, an ion-exchange resin, a solvent, and the like can provide a coffee beverage richer in the taste and aroma of coffee itself.

Here, examples of the drink include those called liquid drinks offered commercially in cans or so-called PET bottle containers. Examples of the above-described powdered drink mix include instant coffee mixes, instant tea mixes, and instant fruit juice drink mixes. Examples of the instant coffee include so-called soluble powdered coffees each obtained by extracting a roasted and ground coffee with boiling water, followed by removing water from the resultant extract using a spray- or freeze-drying method; examples of the coffee mix drink include a drink in which sugar, creaming powder, and the like are added to, and mixed with, a soluble powdered coffee.

For solving the above-mentioned problems, the present inventors have used a drink having the effect of treating, preventing, or improving diabetes or diabetic complications, comprising the oligosaccharides in each of which 2 to 10 (inclusive) mannose molecules are linked together obtained by the above-described method to study the glucose tolerance- and hyperglycaemia-improving effect thereof. In addition, an effect thereof on blood glucose level in humans has been also examined. As a result, the present inventors have found that the drink exerts the effect of improving glucose tolerance and hyperglycaemia in animals and the effect of lowering blood glucose level in humans, thereby accomplishing the present invention.

The food or drink used for treating, preventing, or improving diabetes or diabetic complications according to the present invention comprises mannooligosaccharides having these effects and can be produced specifically by hydrolyzing mannan. The effect of treating, preventing, or improving diabetes or diabetic complications can be expected by the ingestion of the food or drink used for treating, preventing, or improving diabetes or diabetic complications according to the present invention on a daily basis. Coffee beans or coffee extracted residues can be, for example, used as a raw material for the composition used for treating, preventing, or improving diabetes or diabetic complications according to the present invention. Thus, according to the present invention, a composition having the effect of treating, preventing, or improving diabetes or diabetic complications can be prepared from coffee extracted residues previously treated as a waste to ingest the composition together with a food and drink or the like; therefore, the present invention is a very useful invention in view of reuse of waste resources as well as health improvement.

A composition having the effect of treating, preventing, or improving diabetes or diabetic complications according to the present invention was used to examine the glucose tolerance- and hyperglycaemia-improving effect thereof in animals and an effect thereof on blood glucose level in humans. The present Examples specifically describe embodiments of the present invention and are not intended to be limiting with respect to the scope of the invention.

### Example 1

Preparation of mannooligosaccharides. A roasted and ground coffee obtained by an ordinary method was extracted with a commercially used percolation system and the remaining coffee extraction residue was used.

To facilitate the feeding of the coffee extraction residue into a reactor, the residue was first ground into a particle size of about 1 mm. A slurry composed of water and the ground product, having a total solids concentration of about 14% by weight, was then prepared and heat-treated in a 4-m thermal plug flow reactor. The slurry was pumped together with high-pressure steam at a speed corresponding to a residence time of 8 minutes into a plug flow reactor, and kept at about 210° C. using a 6.35 mm diameter orifice. Subsequently, the slurry was spouted at atmospheric pressure to quickly stop the reaction. The resultant slurry was filtered to separate a solution containing soluble solids from insoluble solids. This soluble solids-containing solution was decolorized using active carbon and an adsorbent resin and further desalted with an ion-exchange resin, followed by concentration and drying to provide, at a yield of 14%, a composition comprising oligosaccharides in each of which 1 to 10 molecules of monosaccharides mainly comprising mannose are linked together.

The DP distribution of oligosaccharides contained in the composition, thus obtained, having the effect of treating, preventing, or improving diabetes or diabetic complications is, for example, as follows: DP 1; 2.4%, DP 2; 26.6%, DP 3; 20.2%, DP 4; 17.8%, DP 5; 10.9%, DP 6; 8.9%, DP 7; 6.0%, DP 8; 3.6%, DP 9; 1.9%, and DP 10; 1.7%, where the content of mannose residues in the sugar chain is 90%, however, the DP distribution and the content of mannose residues in the sugar chain can have various values depending on the conditions of hydrolysis. The oligosaccharides in this composition could include, for example, mannose as an oligosaccharide having DP 1, mannobiose as one having DP 2, mannotriose as one having DP 3, mannotetraose as one having DP 4, mannopentaose as one having DP 5, mannohexaose as one having DP 6, mannoheptaose as one having DP 7, mannooctaose as one having DP 8, mannonononaose as one having DP 9, and mannodecaose as one having DP 10, where these mannooligosaccharides had β-1,4-glycoside bonds. The mannooligosaccharides thus obtained were used to perform the following experiment.

### Example 2

Verification experiment on effect of administration of mannooligosaccharides on glucose tolerance. Female ICR mice were used in the experiment. The mice were preliminarily observed for one week for quarantine and conditioning, and of these mice, individuals having shown no abnormalities in body weight change and general condition were then employed for the experiment. The mice were maintained at controlled temperature and humidity using a 12-hour light and 12-hour dark cycle. A normal diet (CE-2 from Clea Japan, Inc.) was given ad libitum as a feed, and city water was provided ad libitum as a drinking water for the period of quarantine and conditioning. After the preliminary period, the mice were divided into groups of 6 individuals each so that the mean body weights of groups were approximately equal. The group structure consisted of 4 groups (i.e., a normal diet group, a high-fat diet group, a 3% mannooligosaccharides-containing high-fat diet group, and a 9% mannooligosaccharides-containing high-fat diet group). As feeds, the same diet as that for the preliminary conditioning was given to the normal fat diet group and a high-fat diet having the following composition to the high-fat diet group. The normal diet was the CE-2 diet. The high-fat diet composition was 40 weight % tallow, 10 weight % corn starch, 9 weight % sugar, 1 weight % AIN76TM-mixed vitamins, 4 weight % AIN76TM-mixed minerals, and 36 weight % casein. For the mannooligosaccharides-containing high-fat diet groups, mannooligosaccharides are added in amounts of 3% by weight and 9% by weight each to the above-described high-fat diet. The increment was adjusted using casein.

A glucose tolerance test by administration of glucose was performed at the 12th week of feeding. More specifically, the mice were fasted for 16 hours, followed by oral administration of glucose (0.8 g/individual). Blood was collected before administration (i.e., time zero) from the tail and then at 60, 120, and 180 minutes after administration. The blood glucose levels for the various blood samples were then determined.

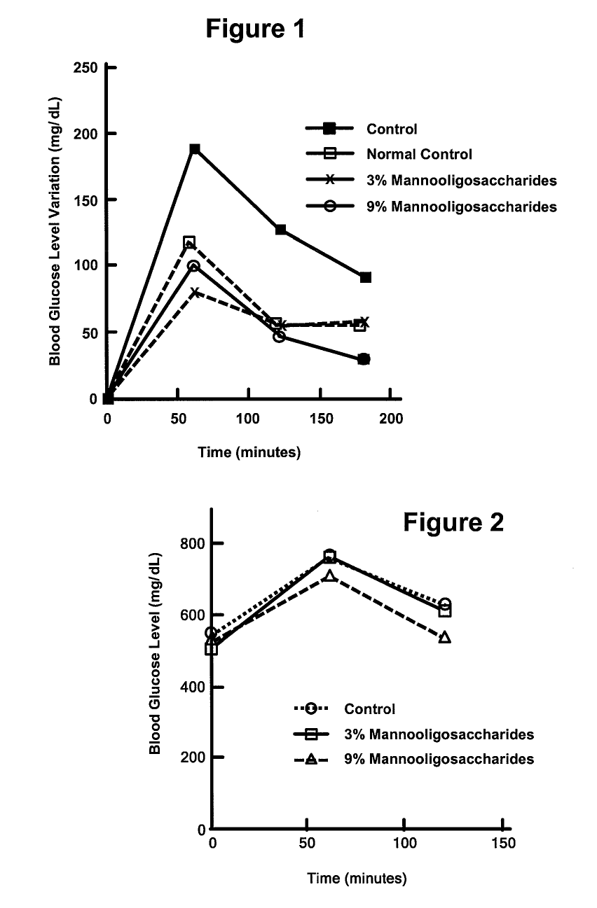

The results of the glucose tolerance test are shown in FIG. 1. The high-fat diet group (control) showed a significantly higher value of variation in blood glucose level than the normal diet group (normal control) at 60 minutes, and maintained high values thereof throughout the experiment. The variation was probably not affected by glucose in the diet because this experiment was performed by glucose administration after 16 hours of fasting, thereby suggesting that an abnormal glucose metabolism is present in the high-fat diet group. Obesity has been found to induce hypoactivity of insulin as a hormone involved in glucose metabolism (i.e., insulin resistance). Thus, it was probable that the high-fat diet group showed the high blood glucose concentration because of reduced insulin action due to obesity.

In contrast, blood glucose concentrations in the 3% mannooligosaccharides-containing high-fat diet group and the 9% mannooligosaccharides-containing high-fat diet group underwent transitions to the same extent as that in the normal diet group, and were significantly lower than the concentration in the high-fat diet group (p<0.05 or p<0.01). These mannooligosaccharides-containing high-fat diet groups had normal glucose metabolism despite the ingestion of the high-fat diet, showing that the administration of mannooligosaccharides improved glucose tolerance (FIG. 1).

### Example 3

Verification experiment on effect of administration of mannooligosaccharides on blood glucose level in diabetic rats. Male Wistar rats (from Charles River Laboratories Japan, Inc.) were purchased and preliminarily maintained for one week in a room controlled at a temperature of 23° C. and a humidity of 60%. Healthy rats from this group were then used for the experiment. Streptozotocin (65 mg/kg) was intraabdominally administered to the rats to prepare diabetic model rats. Individuals having blood glucose levels of 300 mg/dl or more were defined as diabetic rats and divided into groups of 5 individuals each using blood glucose level as an indicator. The group structure consisted of 3 groups: a control group, a 3% mannooligosaccharides treatment group, and a 15% mannooligosaccharides treatment group. A diet (CE-2 from Clea Japan, Inc.) and city water were given ad libitum during the test period for all groups. A 3% or 15% solution of mannooligosaccharides was forcibly administered orally thrice daily (6 ml/day) to each of the test groups. An equivalent volume of distilled water was forcibly administered orally to the control group at the same rate. The test period was set to 28 days; blood was collected under conditions of non-fasting at the 14th day of the test period and under fasting at the 28th day of the test period to determine blood glucose level. In addition, a glucose tolerance test was performed in the same manner as in Example 1 during the testing period.

The results of the blood glucose level determination and the glucose tolerance test are shown in Table 1 and FIG. 2, respectively. The blood glucose level during non-fasting at the 14th day was significantly lower in the 15% mannooligosaccharides treatment group than that in the control group (p<0.01). In addition, the blood glucose level during fasting at the 28th day was significantly lower in 3% and 15% mannooligosaccharides treatment groups than that in the control group (p<0.001). In the glucose tolerance test, blood glucose tended to stay at lower levels in the 15% mannooligosaccharides treatment group than that in the control group. These results suggest that the administration of mannooligosaccharides enables the improvement of diabetic hyperglycaemia and glucose tolerance to be expected.

TABLE 1
Effect of mannooligosaccharides on blood glucose level in rats*

| | Before Experiment | 14th Day | 28th Day |
|---|---|---|---|
| | (Non-fasting) | (Non-fasting) | (Fasting) |
| Control | 479.3 ± 22.7 | 486.5 ± 6.0 | 197.2 ± 7.0 |
| 3% Mannooligo- | 473.7 ± 7.0 | 481.4 ± 37.2 | 95.4 ± 5.0*** |
| saccharides | | | |
| 15% Mannooligo- | 483.0 ± 8.4 | 405.8 ± 9.0** | 56.1 ± 4.4*** |
| saccharides | | | |

*Each value represents mean ± standard error (n = 5).
**Significant different to control at p < 0.01.
***Significant different relative to control at p < 0.001.

### Example 4

Verification experiment on effect of administration of mannooligosaccharides on blood glucose level in humans. The effect of the drinking of a mannooligosaccharides-containing drink on blood glucose level was examined in humans.

Persons having elevated blood glucose levels but within the normal range were selected as subjects (n=5). A liquid coffee was used as a test drink; the coffee was prepared by adding water to a concentrated coffee extract, mannooligosaccharides (3 g/300 ml), and an artificial sweetener for dilution before UHT sterilization, and charged into a 900-ml PET bottle. The amount and period of ingestion were 900 ml per day and 4 weeks, and blood was collected before ingestion (0th day) and at the 4th week of ingestion to determine blood glucose level. A conventional liquid coffee (trade name: Blendy Bottle Coffee from Ajinomoto General Foods, Inc.) was used as control over a somewhat longer period of 12 weeks.

The results are shown in Table 2. The fasting blood glucose level at the 4th week (90.0±3.0 mg/dL) was significantly lowered compared to the fasting blood glucose level before ingestion (96.0±3.6 mg/dL) (p<0.05). These results suggest that the administration of mannooligosaccharides enables the lowering of blood glucose level to be expected.

TABLE 2
Effect of mannooligosaccharides on blood glucose level

| | Blood Glucose Level (mg/dL)* | |
|---|---|---|
| | Initial | End of |
| | (i.e., before Experiment) | Experiment** |
| Control | 95.0 ± 3.65 | 94.6 ± 3.53 |
| Mannooligosaccharides | 96.0 ± 3.59 | 90.0 ± 30.3*** |

*Each value represents mean ± standard error (n = 5).
**Control and inventive samples were tested over a 4 and 12 week period, respectively.
***Significant different relative to initial blood glucose level at p < 0.001.

The control group did not show such lowered blood glucose levels.

What is claimed is:

1. A composition for treating, preventing, or improving diabetes or diabetic complications, comprising oligosaccharides at a concentration of 0.15 to 10% by weight wherein the oligosaccharides include mannose molecules linked together with a degree of polymerization of 2 to 10 (inclusive).

2. The composition of claim 1, wherein the degree of polymerization is 2 to 6 (inclusive).

3. The composition of claim 1, wherein the oligosaccharides are obtained by subjecting mannan to hydrolysis treatment.

4. The composition of claim 3, wherein the mannan is obtained from coffee beans, coffee extracted residues, or mixtures thereof.

5. The composition of claim 2, wherein the oligosaccharides are obtained by subjecting mannan to hydrolysis treatment.

6. The composition of claim 5, wherein the mannan is obtained from coffee beans, coffee extracted residues, or mixtures thereof.

7. The composition of claim 1, wherein the oligosaccharides are β-1,4-mannooligosaccharides.

8. The composition of claim 4, wherein the oligosaccharides are β-1,4-mannooligosaccharides.

9. The composition of claim 6, wherein the oligosaccharides are β-1,4-mannooligosaccharides.

10. The composition of claim 1, wherein the composition is a beverage selected from the group consisting of a liquid coffee drink, a liquid tea drink, a liquid fruit juice drink, an instant coffee, a powdered coffee mix drink, a powdered tea drink, and a powdered fruit juice drink.

11. The composition of claim 4, wherein the composition is a beverage selected from the group consisting of a liquid coffee drink, a liquid tea drink, a liquid fruit juice drink, an instant coffee, a powdered coffee mix drink, a powdered tea drink, and a powdered fruit juice drink.

12. The composition of claim 6, wherein the composition is a beverage selected from the group consisting of a liquid coffee drink, a liquid tea drink, a liquid fruit juice drink, an instant coffee, a powdered coffee mix drink, a powdered tea drink, and a powdered fruit juice drink.

13. A method for treating, preventing, or improving diabetes or diabetic complications in a subject, said method comprising administering to the subject an effective amount of a composition comprising oligosaccharides at a concentration of 0.15 to 10% by weight wherein the oligosaccharides include mannose molecules linked together with a degree of polymerization of 2 to 10 (inclusive).

14. The method of claim 13, wherein the administration to the subject is orally and the effective amount of the composition provides 0.1 to 40 g of the oligosaccharides per day to the subject.

15. The method of claim 14, wherein the composition is a food or beverage.

16. The method of claim 15, wherein the degree of polymerization is 2 to 6 (inclusive).

17. The method of claim 14, wherein the oligosaccharides are obtained by subjecting mannan to hydrolysis treatment.

18. The method of claim 17, wherein the mannan is obtained from coffee beans, coffee extracted residues, or mixtures thereof.

19. The method of claim 17, wherein the oligosaccharides are β-1,4-mannooligosaccharides.

20. The method of claim 14, wherein the composition is a beverage selected from the group consisting of a liquid coffee drink, a liquid tea drink, a liquid fruit juice drink, an instant coffee, a powdered coffee mix drink, a powdered tea drink, and a powdered fruit juice drink.

21. The method of claim 20, wherein the oligosaccharides are obtained by subjecting mannan to hydrolysis treatment.

22. The method of claim 21, wherein the mannan is obtained from coffee beans, coffee extracted residues, or mixtures thereof.
